(19) United States
(12) Patent Application Publication
(10) Pub. No.: US 2021/0241909 A1
(43) Pub. Date: Aug. 5, 2021

(54) METHOD AND A SYSTEM FOR EVALUATING TREATMENT STRATEGIES ON A VIRTUAL MODEL OF A PATIENT

(71) Applicant: KONINKLIJKE PHILIPS N.V., Eindhoven (NL)

(72) Inventors: MURTAZA BULUT, EINDHOVEN (NL); CORNELIS PETRUS HENDRIKS, EINDHOVEN (NL); LIEKE GERTRUDA ELISABETH COX, EINDHOVEN (NL); VALENTINA LAVEZZO, HEEZE (NL); HERMAN GUILLERMO MORALES VARELA, SURESNES (FR)

(21) Appl. No.: 17/095,299

(22) Filed: Nov. 11, 2020

Related U.S. Application Data
(60) Provisional application No. 62/969,304, filed on Feb. 3, 2020.

(51) Int. Cl. G16H 50/20; A61B 34/10; G16H 50/50; G16H 10/60; G16H 70/20; G16H 70/60; G16H 50/70

(52) U.S. Cl. CPC G16H 50/20; A61B 34/10; G16H 50/50; G16H 10/60; A61B 2034/105; G16H 70/60; G16H 50/70; A61B 2034/104; G16H 70/20

(57) ABSTRACT

A method of assessing the impact of a certain treatment strategy for a patient on a digital twin or virtual model of that patient. It is recognized that the impact on a virtual twin could determine whether or not a treatment strategy is selected, as clinicians are becoming increasingly reliant on virtual twins to perform long-term monitoring of a patient's condition.

### FIELD OF THE INVENTION

The present invention relates to the field of virtual models for patients, such as digital twins, and, in particular, to treatment strategies using virtual models.

### BACKGROUND OF THE INVENTION

There is a current practice in the healthcare profession for a clinician to select or provide a treatment strategy that has the highest chance of improving a patient's health or quality of life. However, selecting an appropriate treatment strategy is a difficult process, and there is a desire to improve the amount of information available to a clinician to aid them in making an appropriate clinical decision.

One method of providing the clinician with additional information and/or guidance is through the use of a “virtual model”, “digital model”, “digital twin” or “virtual twin” of the patient.

A virtual model is a digital representation of at least part of the anatomy and/or a bodily/psychological process of the patient, i.e. a biological model. The virtual model processes input data, which may provide characteristics or measured parameters of the patient, to generate output data. The output data may comprise other predicted characteristics of the patient.

For example, a virtual model of a circulatory system may be able to estimate blood flow information based on blood pressure and/or body measurements of the subject. In another example, a treatment strategy for a subject may be recommended by a virtual model (albeit only acted upon when a clinician agrees). Other methods of exploiting a virtual model or digital twin would be well known to the skilled person.

Use of a digital twin or virtual model is an ongoing trend, and it has been found that there is an increasing reliance, by clinical staff, on the information produced by a digital twin or virtual model.

There is therefore an ongoing desire to improve the information provided by a digital twin or virtual model, and to ensure that the provided information continues to be reliable, accurate, consistent, appropriate and complete.

### SUMMARY OF THE INVENTION

The present disclosure is directed to inventive methods and systems for predicting and evaluating the effect of a potential treatment strategy on a virtual model of a patient.

Generally, in one aspect, the invention focuses on a computer-based method including the steps of: obtaining a virtual model of a patient, the virtual model comprising a digital representation of at least part of the anatomy and/or a bodily/psychological process of the patient, wherein the virtual model uses input data, providing information on characteristics of the patient, to generate output data; receiving treatment information, the treatment information indicating a potential treatment strategy for the patient; processing the treatment information and the virtual model to predict an effect of the potential treatment strategy on the virtual model of the patient; and generating, based on the processing, effect information that indicates the predicted effect of the potential treatment strategy on the virtual model of the patient.

The invention stems from the realization that a treatment strategy could affect the ability of a virtual model (or “digital twin”) to accurately generate output data for use by a clinician. For example, input data for a virtual model could be rendered inaccurate or unobtainable by certain medications or treatment strategies, resulting in output data generated by a virtual model being inaccurate, non-representative of the patient and/or completely absent.

The inventors have recognized and appreciated that a clinical decision, such as deciding how to treat a patient, could be improved by assessing the impact of a potential treatment strategy on the virtual model. As virtual models are increasingly important in monitoring the status of a patient, the inability to rely upon use of a virtual model could have a significant effect on the future medical care of the patient (e.g. as selection of future treatment strategies could be made more difficult if the clinician relies upon potentially unreliable or missing data from a virtual model).

The inventors have therefore recognized and appreciated that a credible assistance would be provided when making a clinical decision, such as deciding how to treat a patient, if information on how the virtual twin is affected by a proposed treatment strategy could be provided. In particular, the long-term health of the patient is causally linked to an ability of a virtual model to continue to (accurately, reliably and consistently) predict output data of the patient, as such output data is becoming increasingly relied upon by clinicians when making their clinical decision.

The present invention, in its various aspects and embodiments, therefore proposes to generate new information, previously unavailable to the clinician, to aid the clinician in making an appropriate clinical decision to improve the long-term prognosis of their patient.

Preferably, the effect of the potential treatment strategy is the effect of any element of the treatment strategy that does not mitigate the underlying condition that the treatment strategy is attempting to address. This may include, for example, side-effects of the treatment strategy, a location at which the treatment strategy takes place, timing of the treatment strategy and so on. This enables the proposed method to enable monitoring of whether the virtual model is able to accurately track information for monitoring the subject undergoing the treatment strategy (i.e. whether the proposed treatment strategy mitigates the condition/disease of the subject).

The effect of the potential treatment strategy may be the effect of one or more side-effect(s) of the potential treatment strategy, i.e. effects to the virtual model (e.g. the input/output data or the processing performed by the virtual model) that do not represent an intended effect of applying the treatment strategy to the patient.

The method may further include a step of visually displaying the effect information to a user, i.e. generating a visual representation of the effect information. This aids the user by presenting additional information, previously unavailable to them, for aiding in the selection of a treatment strategy. The display may be provided via a user interface or the like.

In some embodiments, the step of receiving treatment information comprises using the virtual model to generate the treatment information.

Thus, the virtual model may be adapted to recommend a treatment strategy (by generating treatment information) for the patient, i.e. based on input data associated with the patient. The step of recommending a treatment strategy may be performed by a “plug-in” for the virtual model (i.e. an additional processing element or module), to process an output of a digital representation of a part of the anatomy and/or bodily/psychological process in order to recommend a treatment strategy. Methods of recommending a treatment strategy are well known to the skilled person.

The proposed approach enables an automated method for assessing whether a treatment strategy proposed by the virtual model is suitable for future use of the virtual model. This information could, for example, be exploited to improve or adapt the virtual model so that recommended treatment strategies are suitable for future use of the virtual model.

It is emphasized and acknowledged that, for at least ethical reasons, the final decision on any treatment strategy to be performed on a patient is made by a clinician. Using a virtual model to generate or recommend a treatment strategy can aid a clinician in making their selection of a suitable treatment strategy.

In some embodiments, the step of processing the treatment information and the virtual model comprises predicting the effect of the treatment information on the input data used by the virtual model, to thereby predict the effect of the potential treatment strategy on the virtual model.

The present inventors have recognized that certain treatment strategies will affect the input data available to the virtual model. For example, a particular treatment strategy may result in a certain feature of input data (e.g. heart rate information) being unreliable, or may result in another feature of input data (e.g. electroencephalography information) becoming unavailable—e.g. if the treatment strategy recommends that the subject be treated at home or in a clinical setting without the ability to obtain electroencephalography information.

Thus, determining the effect of the treatment information on the input data enables an assessment of the utility or usefulness of the virtual model in the future (i.e. during treatment).

The step of predicting the effect of the treatment information on the input data may comprise predicting: the effect of the potential treatment strategy on the availability of at least some of the input data; the effect of the potential treatment strategy on noise or error-rate in at least some of the input data; and/or whether the potential treatment strategy will introduce new features to the input data.

The step of processing the treatment information and the virtual model optionally comprises: using the virtual model to generate first output data based on the input data; modifying the input data based on the treatment information; using the virtual model to generate second output data based on the modified input data; and comparing the first output data and the second output data to predict the effect of the treatment information on the virtual model.

This provides a simple and effective method of determining an accuracy or effect of the potential treatment strategy on the virtual model, by effectively modelling how adjusting the input data for the virtual model (based on the treatment information) would affect the output of the virtual model. This enables an assessment as to whether the virtual model continues to produce a same output (i.e. remains accurate) even if input data is modified.

Comparing the first output data to the second output data may comprise directly comparing values of the first output data to corresponding values of the second output data (e.g. using a mean-squared error approach) to determine an effect of the potential treatment strategy on the output data.

However, this step may include comparing other characteristics, such as one or more performance metrics, of the first and second output data in order to determine an effect of the potential treatment strategy on the output data. For example, a size of the confidence interval, variance of output data, percentage of missing output data and/or time to generate output data could be determined for each of the first and second output data. This information can then be compared to predict the effect of the treatment information on the virtual model.

In embodiments, the step of comparing the first output data to the second output data comprises predicting an accuracy of the virtual model, after the treatment strategy is performed on the patient, using the first output data and the second output data; and the step of generating effect information comprises outputting the determined accuracy as the effect information.

It is important for the virtual model to remain accurate throughout treatment of the subject. The proposed approach enables the accuracy of the virtual model during the proposed treatment to be predicted, to thereby objectively assess the effect of the proposed treatment strategy on the virtual model in a numeric and comprehensible manner.

In at least one embodiment, the first output data comprises a first recommended treatment strategy and the second output data comprises a second recommended treatment strategy.

Recommending a treatment strategy typically requires combination of different data elements from the virtual model, so that comparing treatment strategies recommended by a virtual model enables a new method of assessing the effect of the proposed treatment strategy on the overall (or a large portion of the) virtual model, e.g. rather than only certain elements of the virtual model.

Optionally, the step of processing the treatment information and the virtual model comprises predicting the effect of the treatment information on the step of generating output data performed by the virtual model, to thereby predict the effect of the potential treatment strategy on the virtual model.

It is recognized that certain treatments may affect the ability of the output data to accurately model the patient. For example, a surgery could affect the geometry of the patient, meaning that a previously accurate virtual model no longer accurately represents the patient's anatomy or bodily function.

Thus, assessing the effect of the treatment information on the step of generating output data (i.e. processing the input data to generate the output data) facilitates an alternative approach to assessing an accuracy or relevance of the virtual model during potential future treatment.

In embodiments, the step of processing the treatment information and the virtual model comprises predicting the effect of the treatment information on the output data generated by the virtual model, to thereby predict the effect of the treatment information on the virtual model.

It is recognized that treatment information may affect the availability of the output of the virtual model. By way of example only, some virtual models may require potentially complex user interfaces in order to present all of their output data (e.g. with only a limited subset being available otherwise). If a treatment strategy recommends being treated at home, or in a low-tech clinical setting, then not all output data may be available to the clinician.

Thus, assessing the effect of the treatment information on the output data provides an alternative approach to assessing the effect of the potential treatment strategy on the virtual model, and takes into account new features that would affect the availability of the output data (produced by the virtual model) during the course of potential future treatment.

In some embodiments, the step of processing the treatment information and the virtual model comprises: predicting, based on the treatment information, a physiological effect of the potential treatment strategy on one or more physiological parameters of the patient or on the monitoring of one or more physiological parameters; and determining, based on the predicted physiological effect and the virtual model, the effect of the potential treatment strategy on the virtual model of the patient.

Different treatment strategies may affect the physiological parameters of the patient (e.g. increase blood pressure, reduce heart rate) which would hinder the ability of the virtual model to obtain accurate input data for generating output data. Some treatment strategies would affect the ability of a sensor to accurately sense certain physiological parameters. For example, certain drugs may interfere with signals sensed by a sensor.

The step of processing the treatment information and the virtual model may comprise: predicting, based on the treatment information, a behavioral and/or psychological effect of the potential treatment strategy on the patient and/or caregivers; and determining, based on the predicted behavioral and/or psychological effect and the virtual model, the effect of the potential treatment strategy on the virtual model of the patient.

A treatment strategy may affect a behavior of the patient or caregiver, which may affect the input data provided to the virtual model that the proposed embodiment takes into account. For example, a certain treatment strategy (e.g. treatment at home) may cause monitoring of the patient (for obtaining input data) to be taken less regularly than required for a particular virtual model to accurately generate output data.

Thus, the behavioral or psychological effect of the potential treatment strategy can affect the ability of the virtual model to accurately and reliably generate output data during potential future treatment of the patient.

The computer-based method may further comprise a step of receiving patient data, the patient data providing information on the patient, and wherein the step of processing the treatment information and the virtual model comprises processing the treatment information, the patient data and the virtual model to predict a combined effect of the potential treatment strategy and the patient data on the virtual model of the patient.

It has been recognized that patient data will also affect the ability of the virtual model to accurately model the patient in the future. For example, patient data may indicate an intent of the patient to visit somewhere at high altitude, which could affect the ability of the virtual model to accurately model the patient's anatomy or bodily function. The effect of patient data may be particularly pronounced for different treatment strategies, e.g. the effects of a treatment strategy may differ between different types of patient (e.g. between males and females, or between neo-natal and geriatric patients). This embodiment takes account of the combined effect of the patient data and the treatment strategy in order to improve the determination of the overall effect on the virtual model of the patient.

Generally, in another aspect, there is proposed a computer-based method of generating comparative information for comparing treatment strategies for a patient, the computer-based method comprising: obtaining a virtual model of a patient, the virtual model comprising a digital representation of at least part of the anatomy and/or a bodily/psychological process of the patient, wherein the virtual model uses input data, providing information on characteristics of the patient, to generate output data; obtaining treatment information for each of a plurality of different possible treatment strategies; evaluating, for each treatment strategy, the effect of each possible treatment strategy on a virtual model by performing any appropriate herein described method, using the treatment information with the said treatment strategy; and generating comparative information based on the effect information generated by the step of evaluating each treatment strategy.

For example, the step of generating comparative information for comparing treatment strategies may include clustering the treatment strategies into two or more sets/groups/clusters based on the effect information of each treatment strategy. In another example, treatment strategies may be compared with the pros and cons of each treatment strategy indicated. In yet another example, specific performance parameters of each treatment strategy may be generated for enabling a comparison of the treatment strategies.

In some embodiments, the step of generating comparative information for comparing treatment strategies comprises ranking the treatment strategies based on the effect information of each treatment strategy. However, other approaches may also be adapted from any herein described method, such as clustering the treatment strategies. Ranking the different treatment strategies based on the preference of the patient or the clinician, provides the clinician with additional information in order to select an appropriate treatment strategy for the patient.

In some embodiments, the step of processing the treatment information and the virtual model for each available treatment may predict and indicate whether a certain treatment strategy alters the virtual model itself, thus the treatment strategy is not suitable to use with the model. This information may be considered when generating comparative information for comparing treatment strategies.

In some embodiments, the computer-based method to compare treatment strategies comprises a step of processing the treatment information on the virtual model that may also predict whether a certain treatment strategy alters the input data available from the patient. The step of processing of the treatment information for each available treatment using the treatment information on the virtual model may optionally predict and indicate whether the potential treatment strategy alters the availability of at least some input data of the patient; and/or it may predict the effect of the potential treatment strategy on the noise or error rate in some data; and/or whether the potential treatment strategy will introduce new features to the input data.

In some embodiments, a computer-based method to compare treatment strategies for the patient that comprises the step of processing the treatment information and the virtual model optionally comprises: using the virtual model to generate first output data based on the input data; modifying the input data based on the treatment information; using the virtual model to generate second output data based on the modified input data; and comparing the first output data and the second output data to predict the effect of the treatment information on the virtual model.

The method determines an accuracy or effect of the potential treatment strategy on the virtual model, by effectively modelling how adjusting the input data for the virtual model (based on the treatment information) would affect the output of the virtual model. Thereby enable an assessment as to whether the virtual model continues to produce a same output (i.e. remains accurate) even if input data is modified. The accuracy of the potential treatment, or the effect of the potential treatment may be considered when generating comparative information for comparing treatments.

Comparing the first output data to the second output data may comprise directly comparing values of the first output data to corresponding values of the second output data (e.g. using a mean-squared error approach) to determine an effect of the potential treatment strategy on the output data.

However, this step may comprise comparing other characteristics, such as one or more performance metrics, of the first and second output data in order to determine an effect of the potential treatment strategy on the output data. For example, a size of the confidence interval, variance of output data, percentage of missing output data and/or time to generate output data could be determined for each of the first and second output data. This information can then be compared to predict the effect of the treatment information on the virtual model for the possible treatment strategies in order to compare them to each other.

In embodiments, the step of comparing the first output data to the second output data comprises predicting an accuracy of the virtual model, after the treatment strategy is performed on the patient, using the first output data and the second output data; and the step of generating effect information comprises outputting the determined accuracy as the effect information for each possible treatment strategy.

It is important for the virtual model to remain accurate throughout treatment of the subject. The proposed approach enables the accuracy of the virtual model during the proposed treatment to be predicted, to thereby objectively assess the effect of the proposed treatment strategy on the virtual model in a numeric and comprehensible manner.

In at least one embodiment, the first output data comprises a first recommended treatment strategy and the second output data comprises a second recommended treatment strategy.

Recommending a treatment strategy typically requires combination of different data elements from the virtual model, so that comparing treatment strategies recommended by a virtual model enables a new method of assessing the effect of the proposed treatment strategy on the overall (or a large portion of the) virtual model, e.g. rather than only certain elements of the virtual model.

Optionally, the step of processing the treatment information and the virtual model comprises predicting the effect of the treatment information on the step of generating output data performed by the virtual model, to thereby generate the comparative information for comparing treatment strategies for a patient.

In embodiments, the step of processing the treatment information and the virtual model comprises predicting the effect of the treatment information on the output data generated by the virtual model, to thereby predict the effect of the treatment information on the virtual model.

It is recognized that treatment information may affect the availability of the output of the virtual model. By way of example only, some virtual models may require potentially complex user interfaces in order to present all of their output data (e.g. with only a limited subset being available otherwise). If a treatment strategy recommends being treated at home, or in a low-tech clinical setting, then not all output data may be available to the clinician.

Thus, assessing the effect of the treatment information on the output data provides an alternative approach to generate comparative information for comparing treatment strategies for each potential treatment strategy on the virtual model, and takes into account new features that would affect the availability of the output data (produced by the virtual model) during the course of potential future treatment.

In some embodiments, the step of processing the treatment information and the virtual model comprises: predicting, based on the treatment information, a physiological effect of the potential treatment strategy on one or more physiological parameters of the patient or on the monitoring of one or more physiological parameters; and determining, based on the predicted physiological effect and the virtual model, the comparative information for comparing treatment strategies for the patient on the virtual model of the patient.

The computer-based method of generating comparative information for comparing treatment strategies for a patient may further comprise a step of receiving patient data, the patient data providing information on the patient, and wherein the step of processing the treatment information and the virtual model comprises processing the treatment information, the patient data and the virtual model to predict a combined effect of the potential treatment strategy and the patient data on the virtual model of the patient.

This embodiment takes account of the combined effect of the patient data and the treatment strategy in order to improve the determination of the overall effect on the virtual model of the patient.

The computer-based method of generating comparative information for comparing treatments may further compromise a step to display the comparative information (e.g. ranked strategies).

There is also proposed a computer program product comprising computer program code that, when executed on a computing device having at least one processing system, causes the at least one processing system to perform all of the steps of any herein described method. There is also proposed a processing system adapted to: obtain a virtual model of a patient, the virtual model comprising a digital representation of at least part of the anatomy and/or a bodily/psychological process of the patient, wherein the virtual model uses input data, providing characteristics of the patient, to generate output data; obtain treatment information, the treatment information indicating a potential treatment strategy for the patient; process the treatment information and the virtual model to predict an effect of the potential treatment strategy on the virtual model of the patient; and generate, based on the processing, effect information that indicates the predicted effect of the potential treatment strategy on the virtual model of the patient.

Preferably, the processing system is adapted to process the corresponding treatment information and the virtual model for each of the treatment strategies to predict an effect of the treatment information on the patient's virtual model, then generate information that indicates the predicted effect of the treatment information on the virtual model of the patient.

These and other aspects of the invention will be apparent from and elucidated with reference to the embodiment(s) described hereinafter.

### BRIEF DESCRIPTION OF THE DRAWINGS

For a better understanding of the invention, and to show more clearly how it may be carried into effect, reference will now be made, by way of example only, to the accompanying drawings, in which:

FIG. 1 illustrates a method according to an embodiment;

FIG. 2 illustrates another method according to an embodiment; and

FIG. 3 illustrates a method according to another embodiment.

### DETAILED DESCRIPTION OF THE EMBODIMENTS

The invention will be described with reference to the Figures.

It should be understood that the detailed description and specific examples, while indicating exemplary embodiments of the apparatus, systems and methods, are intended for purposes of illustration only and are not intended to limit the scope of the invention. These and other features, aspects, and advantages of the apparatus, systems and methods of the present invention will become better understood from the following description, appended claims, and accompanying drawings. It should be understood that the Figures are merely schematic and are not drawn to scale. It should also be understood that the same reference numerals are used throughout the Figures to indicate the same or similar parts.

The invention provides a method of assessing the impact of a certain treatment strategy for a patient on a digital twin or virtual model of that patient. It is recognized that the impact on a virtual twin could determine whether or not a treatment strategy is selected, as clinicians are becoming increasingly reliant on virtual twins to perform long-term monitoring of a patient's condition.

Indeed, the invention relies on the recognition that information about the effect of a treatment strategy upon a virtual twin provides useful information to a clinician in determining whether the patient can continue to be accurately monitored by a virtual model. Thus, there is a direct connection to the patient's long-term health.

Embodiments can be used, for example, in any clinical setting to determine the impact of a proposed or recommended treatment strategy.

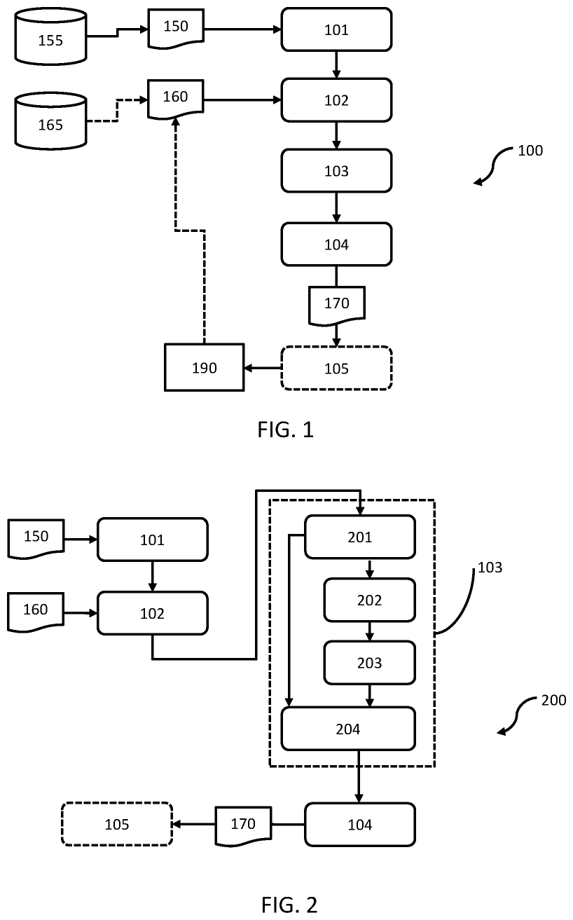

FIG. 1 illustrates a method 100 according to a general concept of the underlying invention.

The method 100 comprises a step 101 of obtaining a virtual model 150 of a patient, which is commonly called a “digital twin”. As previously discussed, a virtual model 150 comprises a digital representation of at least part of the anatomy and/or a bodily/psychological process of the patient. A model of a psychological process of the patient may, for example, model a behavioral process of the subject (e.g. their sleeping patterns, mood variations, stress levels, social activities or physical activity). The virtual model 150 uses input data, providing information on characteristics of the patient, to generate output data.

By way of example only, a virtual model 150 may model the urinary tract of a patient. The model of the urinary tract may receive, as input, urinary data of the subject and provide, as output, a simulation of the urinary tract and/or detection/diagnosis of any problems with the urinary tract (e.g. identifying infection). Example of such urinary data may include (average) volume of urine, glucose level, urinalysis results and so on.

As another example, the virtual model 150 may be of the hemodynamic system in the head, which processes (as input data) central, i.e. bodily, blood pressure information and CT scan information to generate (as output data) blood pressure information and flow distribution in the brain. The virtual model 150 thereby monitors the condition of the patient, e.g. to aid in performing titration treatments, and may provide warnings if necessary (e.g. blood pressure in the brain exceeds a threshold).

Other suitable examples for a virtual model 150 will be apparent to the skilled person, such as a cardiovascular system model, a respiratory system model, a hemodynamic system model, a model of the heart, a model of the lungs, a reproductive system model, a model of the patient's mental status, a model of the patient's anxiety levels; a model of the patient's sleeping behavior; a model of the patient's nutritional intake; a model of the patient's physical activity and so on.

The virtual model 150 may be obtained from a database, data storage arrangement, remote server or memory 155 that stores the virtual model 150.

The method 100 further comprises a step 102 of receiving treatment information 160. The treatment information 160 indicates a potential treatment strategy for the patient. This may comprise, for example, receiving details on a proposed medication regime, proposed surgical procedures, proposed therapeutic treatments, proposed patient activities, proposed dietary restrictions, proposed location for treatment, proposed clinicians for handling the treatment of the patient and so on.

The treatment information 160 may be obtained from a database, data storage arrangement, remote server or memory 165 that stores treatment information 160. In some examples, the treatment information 160 is generated by the virtual model itself. In other examples, the treatment information 160 is selected by a user (e.g. via a user interface 190).

The method 100 comprises, in a step 103, processing the treatment information 160 and the virtual model to predict an effect (or effects) of the potential treatment strategy information on the virtual model of the patient.

The treatment strategy may affect one or more of: the available input data for the virtual model or characteristics thereof (e.g. by making some input data elements unavailable/unreliable or introducing newly available input data elements); characteristics of the processing performing by the virtual model (e.g. a correspondence between the virtual model and the real-life patient) and/or the available output data for the virtual model or characteristics thereof (e.g. by making some output data unavailable, e.g. due to location restrictions, or by affecting the accuracy/reliability of output data). It will be clear that affecting any of these elements will affect the overall virtual model of the patient.

Preferably, the effect of the potential treatment strategy is the effect of any element of the treatment strategy that does not mitigate the underlying condition that the treatment strategy is attempting to address.

The effect of the potential treatment strategy may, for example, be the effect of one or more side-effect(s) of the potential treatment strategy, i.e. effects to the virtual model not attributable to a direct intentional effect of the treatment strategy. By way of example, lithium may be used to treat depression, but could also affect liver function (which could affect a virtual model of the subject's liver).

Other elements of the treatment strategy, that do not mitigate the underlying condition, may be considered, such as a location at which the treatment strategy takes place, timing of the treatment strategy and so on.

One underlying concept of the invention is determining the effect of side-effects of a potential treatment strategy on (characteristics of) the virtual model, to thereby determine whether the virtual model is able to continue accurately monitoring the patient during the course of their treatment.

The step of predicting the effect(s) of the potential treatment strategy on the virtual model of the patient may therefore comprise predicting the effect of any of these previously described elements.

The effect may be physiological (e.g. be a result of a direct effect on the patient or be a physical restriction) and/or psychological (e.g. a behavioral effect on how data is gathered).

Various embodiments for step 103 are envisaged and will be later described.

The method 100 further comprises a step 104 of generating, based on the processing, effect information 170 that indicates the predicted effect of the potential treatment strategy on the virtual model of the patient.

The skilled person will appreciate that this step may be incorporated into step 103. Thus, steps 103 and 104 may be conceptually combined into a single step of processing the treatment information and the virtual model to generate effect information 170 that indicates the predicted effect of the treatment strategy on the virtual model of the patient

The effect information 170 may, for example, comprise a binary/discrete indicator of whether the virtual model remains accurate, reliable, consistent and/or complete for the proposed treatment strategy. In some examples, the effect information 170 comprises a numerical indicator of the accuracy, reliability and/or consistency of the virtual model should the proposed treatment strategy be adopted. In yet other examples, the effect information 170 may comprise a descriptor of the effect of the proposed treatment strategy on the virtual model.

In this way, the method 100 generates effect information 170 that indicates the effect of a potential treatment strategy on the virtual model, which could influence the selection of a treatment strategy in order to maintain/preserve long-term monitoring capability of the patient with the virtual model.

One of more further steps may take place using the effect information 170.

For example, the method 100 may further comprise a step 105 of visually displaying the effect information 170 to a user, i.e. generating a visual representation of the effect information 170. This aids the user by presenting additional information for aiding in the selection of a treatment strategy.

Step 105 may be performed via a user interface 190, such as an augmented reality display, a virtual reality display, a projection, a hologram a (touch-sensitive) screen or display, e.g. a two-dimensional LCD or LED screen. This user interface 190 may comprise, for example, a head-mounted display, a smartphone, a smartwatch, smart glasses, a computer monitor and/or a laptop, amongst other examples.

In some embodiments, the user interface 190 is adapted to provide a visual representation of (the output) of the virtual model. In such examples, the user interface 190 may be further adapted to illustrate the effect information on the virtual representation of the virtual model, e.g. to indicate which elements of the virtual model are incomplete/inaccurate.

For example, if the virtual model is of the hemodynamic system in the head, which generates (as output data) blood pressure information and/or flow distribution in the brain, then the user interface 190 may display a virtual representation of a model of the patient's head, with blood pressure and flow distribution information indicated therein. If the effect information indicates that some of the blood pressure information is inaccurate (e.g. for part of the brain), then this may be indicated within the virtual representation of the model of the patient's head (e.g. identifying that some of the calculated blood pressure information is incorrect/inaccurate using a color indicator).

In some examples, the method 100 may further comprise a step (not shown) of retraining (or further training) or updating the virtual model based on the effect information 170. By way of example, if the effect information 170 indicates that the virtual model is made inaccurate by the treatment option, the virtual model may be automatically retained or updated (e.g. using additional data) in an effort to make it more accurate. The retraining or further training of the virtual model may be performed using population data of a population that have undergone the treatment strategy indicated in the treatment information 160, to refine the virtual model with respect to that treatment strategy. The method 100 may then be repeated using the retrained virtual model (to determine whether the retrained virtual model is suitable for the treatment strategy).

It has previously been described how various embodiments or implementation methods for performing steps 103 and 104 may be used by the skilled person to advantage.

Generally speaking, steps 103 and 104 evaluate the suitability of a potential treatment strategy for virtual model monitoring. The analysis of this suitability may be performed with respect to a population (e.g. based on how a population reacts to (elements of) the potential treatment strategy) or with respect to the patient themselves (e.g. based on historical information on how the patient reacts to (elements of) the potential treatment strategy). The population may be a population of similar subjects to the patient, methods of determining which are well known in the art.

Various, non-exhaustive, scenarios for these steps will now be described. In a first scenario, an effect on the input data for the virtual model is considered; in a second scenario, an effect on the processing performed by the virtual model is considered; in a third scenario, an effect on the output data of the virtual model is considered.

In the first scenario, the treatment information 160 may be analyzed to determine the effect of the proposed treatment strategy on the input data for the virtual model. This can be determined, for example, by consulting literature, biophysical models and/or algorithms, or historical information (such as medical guidelines, research articles, patient records, historical information of the patient, mathematical models, simulation results, machine-generated data and so on) about the treatment strategy to identify effects of the treatment strategy on elements of the input data (e.g. from the literature).

In particular, literature, biophysical models or historical information may be processed to identify the effects of a treatment strategy. The identified effects of a treatment strategy may be compared to input data for the virtual model to determine the effects on the input data for the virtual model, e.g. by identifying elements of the input data that correspond to identified effects of a treatment strategy or identifying elements that become newly available as input data due to a particular treatment strategy.

The literature or historical information may relate to patient-specific data (e.g. a patient's medical record may indicate the response of the patient to a certain treatment) or may relate to population information (e.g. the response of a population to a certain treatment).

In particular embodiments, the step 103 may comprise determining the effect of the potential treatment strategy on the availability of at least some of the input data, the effect of the potential treatment strategy on noise or error-rate in at least some of the input data and/or whether the potential treatment strategy introduces new features to the input data.

The determined effect on the input data may be physiological/biological (e.g. a side effect of a prescribed drug) or behavioral/psychological (e.g. reduced monitoring compliance by a patient due to fatigue/mood following chemotherapy). Such information may be derived from literature, biophysical models, or historical information of the patient (or their peers).

In some embodiments of the first scenario, any determined effects on the input data are output as the effect information 170 (e.g. “some input data for Virtual Model A will be made unreliable” or “Virtual Model B will have insufficient data to generate output data”). This may act as a descriptor of the effect of the proposed treatment strategy on the virtual model.

In other embodiments of the first scenario, some further processing is performed on the determined effects on the input data to generate effect information 170 indicating the predicted effect of the potential treatment strategy on the patient.

It will be appreciated that an effect of a treatment strategy on the input data is propagated through the virtual model, so that it also affects the output data. One potential model could therefore modify existing input data, e.g. by introducing estimated modifications based on the determined effect on the input data, and evaluate the output of the virtual model.

In other words, existing input data (e.g. obtained before any potential treatment is applied) may be modified based on the potential treatment. The modified input data may be processed by the virtual model to produce modified output data. The modified output data may be compared to the original output data (which was generated by the virtual model based on the existing, unmodified input data) to determine an effect of the potential treatment strategy on the output data, and therefore the overall virtual model.

In some examples, the accuracy of the virtual model may be numerical calculated, e.g. by comparing the modified output data to the original output data, using standard approaches such as mean-squared error, correlation coefficient, similarity analysis, regression analysis or other error-quantifying or comparative analysis approaches. This provides an objective value indicative of the predicted effect of the treatment strategy on the virtual model.

Other methods of generating such an objective value will be known to the skilled person.

The precise modifications performed on the existing input data will depend upon the predicted effect of the treatment information 160 on the input data. Exemplary modifications may comprise: removing part of input data (to test the effect of missing data, as may be caused by a particular treatment option); introducing noise/errors in the input data (to test the effect of noise that may be caused by a particular treatment option); and/or introducing new data elements to the input data that was not been present in the building of the virtual model (to test the effect of new data, which may be made available by a particular treatment option).

Consider an example in which urine data forms a portion of the input data for a urinary tract virtual model which generates (as output data) a diagnosis of the patient. A certain treatment option (e.g. using non-vitamin K oral anticoagulants as blood thinners) for the patient may have a known side-effect of increasing the risk of blood in the urine. This side-effect may be identified, and used to modify the input data (e.g. modify the urine data to simulate an increase in blood within the urine). The virtual model may then process the modified input data to generate a modified output data. The modified output data may then be compared to the original output data (generated from the unmodified input data) to determine an effect of the treatment strategy on the virtual model. This may comprise, for example, determining a difference between the original output data and the modified output data. This difference may be used to determine effect information 170 or is itself output as effect information 170.

In another example, it is known that some lithium-based drugs can cause certain ion-sensitive electrode sensors to become unreliable or biased. This may result in some input data being rendered unreliable. The effect of a treatment option, which proposes the use of lithium-based drugs, may be predicted by modelling the effect of the treatment strategy on the input data (e.g. by introducing noise or errors into the relevant input data). A comparison can be made between the original output data (generated from the unmodified input data) and the modified output data (generated from the modified input data) to quantify the effect of the treatment strategy on the virtual model.

In the second scenario, the treatment information 160 and virtual model are processed to identify an effect of the potential treatment strategy on the processing (of input data) performed by the virtual model.

Certain treatment strategies, such as surgery or therapeutic treatments, may alter the physical geometry of a patient. For example, a heart surgical procedure (e.g. inserting a stent or performing bypass surgery) would alter the geometry of the heart. Altering the physical geometry of the patient would affect the accuracy of the virtual model of the patient.

The effect of the proposed treatment strategy on the characteristics of the virtual model, e.g. an accuracy or “real-life correspondence” of the processing performed by the virtual model, can therefore be determined.

As another example of the second scenario, certain treatment strategies may affect the availability of certain virtual models. For example, some treatment strategies may require large processing resources that are only available at certain locations (e.g. and not available if the patient is to be treated at home) or require licensing rights that are only available for certain treatments or locations (e.g. some virtual models may only be licensed for certain medications).

The effect of the proposed treatment strategy on the availability of the virtual model could therefore be determined and used to determine an effect of the proposed treatment strategy on the virtual model.

In the third scenario, the treatment information 160 and virtual model are processed to identify an effect of the potential treatment strategy on the output data provided by the virtual model.

It is conceivable that a particular treatment strategy may affect the availability of the output data.

For example, a certain treatment strategy may recommend a home-based treatment, at which certain user interfaces, e.g. software or hardware, (for providing output data) are not available. For example, if a full understanding of the output data requires an augmented reality headset (e.g. if the virtual model generates a full visual representation of an anatomical feature of the patient), and the patient only has access to an regular 2-D screen, this may have implications. Thus, the correspondence between availability of output data and treatment strategy is clear.

As another example, a particular treatment strategy may affect the interpretability of the output data (e.g. to a patient). For example, if a treatment strategy for a certain condition is predicted to make the patient physically or mentally unavailable to access, visualize, or process, or understand the data, then the interpretability of the output data to the patient would be affected. As a specific example, consider the scenario in which there is a patient with Parkinson's Disease, who would typically have “on” and “off” periods, where during “off” period they are not able to physically operate. A certain treatment strategy may affect the length, regularity or intensity of a patient's “off” periods, which could be used as a factor for assessing the usability or interpretability of the output data.

Yet another example could be speed of availability of the output data. For example, if some input data is not directly measurable due to a side-effect of a treatment strategy, then it may be still possible to accurately calculate/estimate output data using different input data, at the expense of speed of generating the output data. A delay in generating output data may not be clinically acceptable for certain conditions, such as stroke. A working example would be if a treatment strategy causes a direct respiratory measurement to become unavailable (e.g. if the patient is treated outside of the hospital); in this scenario a PPG signal could be used to derive respiratory information at the expense of speed of generating output data (as it will take additional time to generate suitable input data). Any combination of the examples described in these three scenarios may be employed in various embodiments. In short, any and all aspects of a virtual model may be influenced or affected by the selection of a certain treatment strategy, and the underlying inventive concept is the recognition of these effects and identifying the ability to draw the clinician's attention to such effects.

One example for performing steps 103 and 104 will be made clear with reference to FIG. 2.

FIG. 2 illustrates a method 200 of predicting the effect of a potential treatment strategy on a virtual model of a patient according to a specific embodiment of the invention.

The method 200 comprises the step 101 of obtaining a virtual model 150 of a patient and the step 102 of receiving treatment information 160. The method 200 further comprises the step 103 of processing the treatment information and the virtual model to predict an effect (or effects) of the potential treatment strategy on the virtual model of the patient. The method 200 also comprises the step 104 of generating, based on the processing, effect information 170 that indicates the predicted effect of the potential treatment strategy on the virtual model of the patient.

Here the step 103 can be divided into a number of sub-steps.

A first sub-step 201 comprises using the virtual model to generate first output data based on the input data. A second sub-step 202 comprises modifying the input data based on the treatment information. A third sub-step 203 comprises using the virtual model to generate second output data based on the modified input data. A fourth sub-step 204 comprises comparing the first output data and the second output data to predict the effect of the potential treatment strategy on the virtual model.

The sub-step 201 preferably comprises generating a first recommended treatment strategy based on the (original, unmodified input data). The sub-step 202 preferable comprises using the first recommended treatment strategy to modify the input data (i.e. the first recommended treatment strategy may form the treatment information). The sub-step 203 preferably comprises generating a second recommended treatment strategy based on the modified input data.

Some virtual models are adapted to recommend (one or more) treatment strategies based on input data. This embodiment takes advantage of this capability to compare the effect of a treatment strategy (generated by the virtual model) on the virtual model itself.

In this way, two recommended treatment strategies may be generated and compared. A difference/similarity between the two recommended treatment strategies can define the effect of the original treatment strategy (i.e. the first recommended treatment strategy) on the virtual model. The difference may be determined using any known process, for example, using any suitable error-quantifying or comparative analysis approaches (such as those previously described).

The process performed in sub-step 202 may depend upon limitations that the first recommended treatment strategy may impose on the (input data for the) virtual model, and may include medical/physiological/biological (i.e. treatment) and human/psychological factors. Some example modifications include: predicting which changes (e.g. artefacts, noise, missing data) are to be expected in the input data due to the recommended treatment strategy, and intentionally introduce these changes; and determining what changes are to be expected in the input data due to the condition and behavior of the patient, and intentionally introduce these changes.

The step 104 may comprise generating effect information 170 that indicates or guides the magnitude of the effect of the first recommended treatment strategy on the virtual model. For example, if the two treatment strategies are markedly different, the original (first) recommended treatment strategy may be marked as unsuitable (e.g. in the effect information 170).

The step 104 may comprise generating a sensitivity/robustness score (e.g. uncertainty or bias score) for each treatment strategy to compare the two treatment strategies.

Generating a sensitivity/robustness score may be performed by performing a sensitivity/robustness analysis on each virtual model as modified according to a treatment strategy. This can also be referred to as sensitivity score calculation robustness score calculation, as well as uncertainty analysis or score calculation, or bias analysis or score calculation. Methods of generating a sensitivity/robustness score, such as sensitivity analysis or robustness analysis, will be known to the skilled person, although a description of a suitable example will be hereafter described for the sake of completeness.

There are two main goals in performing sensitivity/robustness analysis, namely: a quantification of uncertainty in the output data (e.g. calculating a confidence interval or statistical parameter (e.g. p value) for a generated output) and an evaluation of how each element of input data contributes to the output data uncertainty (e.g. ordering the inputs elements in terms of their contribution to the variation in the output).

The procedure of achieving these goals can be described in terms of four general steps:
1) Quantify the uncertainty in each element of input data (e.g. ranges, probability distribution). In our case, this can be performed by predicting how the treatment would influence input data. For the prediction, we generally rely on past information, which includes data of the patient physiology, psychology, behavior, and context; as well as information of the sensor settings, specification, use conditions, and context; caregiver psychology, behavior and context; input data storage specification and so on. In general, starting from the selected treatment we determine all factors that may influence the input data, and based on these factors we estimate what and how input data may be influenced.

2) Identify how the output data is to be analyzed (i.e. which elements of the output data are to be taken into account for calculating the sensitivity/robustness score): In our case, we are interested in the treatment related output parameters of the output data. For example, where the virtual model generates a recommended treatment strategy, these could include, type of the selected treatment, as well as duration, number of medication, type of medications, estimated effect of the treatment on the quality of life. Other patient and context related parameters linked to the treatment could be also considered, such as ability of the patient to follow the treatment, the caregiver support that would be necessary and so on. For some of the outputs, the evaluation may result in discrete numbers (e.g. is the selected treatment same or different), while for some other outputs the result is continuous variable (e.g. duration of the treatment).
3) Vary the input, and run the virtual model a number of times, as defined by the method for calculating the sensitivity/robustness score: This will result in a probability distribution for the targeted output parameters (identified in step 2).

4) Calculate performance metrics (e.g. robustness/uncertainty score) based on the outputs generated in the previous step. For example, for a given probability density function (for a selected output) calculate the relevant statistics (e.g. variance) and use these as a robustness score. Multiple outputs can be analyzed at the same time (by means of multi-dimensional probability density functions, or techniques such as Principal component analysis, independent component analysis, etc.). Alternatively, single outputs or group of outputs can be analyzed individually, and the individual scores can be combined later (e.g. by means of weighting function) to generate the final score.

There are many established methods that are generally based on the steps above. Some examples include: one at a time (OAT/OFAT)—which comprises modifying one input at a time; methods based on calculating output derivative (e.g. adjoint modelling or automated differentiation); correlation analysis; regression analysis (e.g. linear regression, logistic regression and/or Kalman filter); variance based methods that make use of probability distributions (e.g. where calculations may involve use of Monte Carlo methods); variogram-based methods and/or emulators (which are data-modelling or machine-learning approaches that involve building a mathematical function (known as emulator) to approximate input/output behavior of the model: e.g. Gausses processes, decision trees, gradient boosting, polynomial chaos expansions, spline approximations).

The above described methods of generating a sensitivity/robustness score may be employed to generate effect information (i.e. the effect information may comprise a sensitivity/robustness score) for use in any embodiment of the invention.

The method 200 may be repeated if a treatment strategy is considered unsuitable (e.g. repeated with a new treatment strategy).

Any above-described approach with reference to FIG. 2 may be adapted for any generic output data (rather than recommended treatment strategies), as would be appreciate by the skilled person. For example, the output data may comprise a calculation of a physiological parameter (e.g. which cannot be directly measured, such as brain blood pressure) or a simulation of a bodily process.

It is not necessary for the assessed virtual model to relate to the treatment strategy. That is, the treatment strategy may be to address a problem in another area (of the human body) compared to the virtual model.

Consider a scenario in which a patient suffers from neurological problems and symptoms (e.g. vascular dementia, dizziness). A virtual model, such as a model of the hemodynamic system, can support a neurologist in managing the disease and symptoms. The input data for such a model may comprise CT scan information and central blood pressure information. The output data for such a mode may comprise the blood pressure and flow distribution in the brain, which is responsible for, or contributes to, the medical problems. The virtual model aid in the monitoring of the patient's condition, helps to titrate treatments, and provides warnings if necessary.

At the same time, this same patient suffers from benign prostatic hyperplasia (BPH), for which he is being treated by an urologist. In a shared decision making with the patient, the urologist may have the option of recommending a treatment strategy that incorporates treating the patient with alpha-blockers, which is a medication relaxing smooth muscles. However, the effect of this potential treatment strategy (intended to treat problem with the urinary tract) may have an effect on the virtual model of a patient's hemodynamic system (used to assess a neurological problem).

In this scenario, the virtual model of the head becomes invalidated, since the alpha-blockers will alter the baseline geometry of the vascular structure in the head due to unknown and uncontrolled vascular extension effects.

Appropriate effect information 170 may then be presented to the urologist (and/or patient), which may be used to decide to not proceed with the alpha-blocker treatment strategy in order to maintain the reliability and usefulness of the virtual model of the head. In other words, the effect information 170 may indicate that a consequence of a treatment incorporating alpha-blockers is the disruption of the neurological disease management.

Thus, information is provided that will aids a clinician in making an important clinical decision in pursuit of maintaining a long-term help of the patient.

It has previously been discussed how the underlying concept of the present invention is to determine the effect of a proposed treatment strategy on a patient, in order to aid in the selection of a treatment strategy that enables accurate, consistent and reliable continued monitoring of the patient.

Further embodiments may determine the impact or effect of other elements or features on the virtual model to further aid in the assessment of how a treatment strategy affects a virtual model. These other elements may include, for example, patient(-specific) data, clinician data and/or environment information.

Thus, in some embodiments, a step of processing the treatment information and the virtual model to predict an effect of the potential treatment strategy on the virtual model of the patient may comprise processing the treatment information, additional information and the virtual model to predict a combined effect of the potential treatment strategy and additional information on the virtual model of the patient.

For example, patient data may be used to improve the determination of the effect of a treatment strategy on a virtual model, e.g. by taking into account patient-specific considerations such as the patient's medical status and/or their behavioral habits. Thus, embodiments may comprise processing the treatment information, the patient data and the virtual model to predict a combined effect of the potential treatment strategy and the patient data on the virtual model of the patient.

Consider a scenario in which a patient indicates an intention to go to an area of high altitude (>3000 m). This intention may be indicated in patient data (e.g. which may comprise a calendar). In this scenario, the patient may be diagnosed with depression, for which a potential treatment option is to treat the patient with lithium. However, it is known (e.g. indicated by literature) that the effectiveness of lithium differs at different altitudes, due to the different number of red blood cells. In the step of processing the treatment information, the patient data and the virtual model, it may therefore be determined that the virtual model will become unreliable for the proposed treatment when the patient moves to high altitudes (e.g. as it may be based upon data collected at low altitudes (e.g. sea-level)). This effect (unreliable virtual model) may be indicated to the clinician in the form of effect information 170 to enable them to make an informed decision on whether to proceed with the proposed treatment (as the virtual model can no longer be accurately used).

In another scenario, a virtual model is adapted to process electrocardiogram (ECG) information (as input data) to predict a risk of arrhythmia or heart failure. The virtual model may be adapted to also use treatment information (as input data) to improve the generation of the output data (e.g. it may take into account the effect of certain medications, as well the electrocardiogram information, on the risk of arrhythmia or heart failure). In this scenario, patient data may indicate that ECG information cannot be reliably obtained for more than a predetermined period of time per day (e.g. due to the patient frequently travelling or being otherwise unavailable). As previously noted, the virtual model may be adapted to account for different treatment options, but may require different amounts of data for these different treatment options to accurately generate the risk value. This information may be used to determine that there may be insufficient ECG information for the virtual model to accurately calculate output data (e.g. a risk factor) for some treatment options, but not others. Thus, a treatment option may be recommended or not recommended depending upon the amount of ECG information that can be gathered (as derived from the patient data).

These examples clearly demonstrates how patient data can be used to improve the predicting on whether/how a treatment strategy would affect the virtual model.

Patient data may be obtained, for example, from real-time patient monitoring devices, electronic medical records (e.g. stored in a database), a user interface (e.g. for inputting a questionnaire or information derived from discussion(s) with the patient, caregivers, family members and/or friends), online activity of the patient (e.g. social media information, search engine histories and the like) and so on.

In another example, clinician data may be used to improve the determination of the effect of a treatment strategy (using that clinician) on a virtual model.

Consider a scenario in which a clinician is known to be forgetful when executing a certain treatment strategy (e.g. frequently forgetting to monitor certain parameters of the patient). If a treatment strategy proposes to utilize this clinician, that it can be predicted that the input data (e.g. for input data that it is under the responsibility of the clinician) will be inaccurate and/or unreliable, which can be used to determine or predict the effect of that particular treatment strategy on the virtual model with improved precision.

In another scenario, a clinician may be forgetful when providing certain medications to a patient. If a treatment strategy proposes to maintain a medication regime, but change a less forgetful clinician for the new clinician, then it can be predicted that the input data will change (e.g. due to the clinician failing to provide the medication, causing a deterioration in the input data). This can be used to more accurately determine or predict the effect of the proposed treatment strategy on the virtual model.

These examples clearly demonstrate how clinician data can be used to improve the determination of the effect of a treatment strategy upon a virtual model of the patient.

Suitable clinician data may be extracted from medical records, clinical practice data, evidence and/or statistics from the literature. For example, clinical practice data may provide reliability estimates for a given clinician. In particular, the available population data (which could include patient, hospital, caregiver and insurance data) can be used to generate predictions of potential undesired behavior by a clinician. Using the estimates of the undesired behavior, it can be predicted or estimated how the input data for a virtual model.

Similar scenarios and examples could be readily constructed for other possible additional information, such as environment information.

Different environments for treating a patient may have different effects on a virtual model, e.g. on the input data available for a virtual model. By way of example, different environments may have different treatment policies, hygiene levels, staff availability and so on. These factors would have an effect on the virtual model (e.g. its accuracy or completeness).

Purely by way of example, consider a scenario in which a first treatment strategy entails treating the patient at a clinical environment in which certain patient monitors are not available. This would affect the availability of (at least a portion) of the input data for a virtual model. This knowledge can be used to determine or predict the effect of the first treatment strategy on the virtual model.

From the foregoing, it is apparent that the underlying inventive concept relates to the prediction of an effect of a treatment option upon a virtual model. Embodiments may also take into account the (compounding) effects of other additional data associated with the patient, a clinician and/or environment in the determination of the effect of the treatment option.

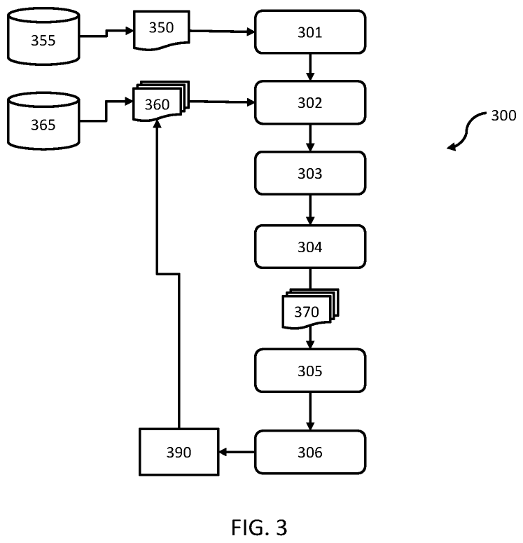

Embodiments of the invention also possible implementations for this underlying inventive concept. One such implementation is hereafter described with reference to FIG. 3.

FIG. 3 illustrates a method 300 according to an embodiment of the invention.

The method comprises a step 301 of obtaining a virtual model 350. As before, the virtual model comprises a digital representation of at least part of the anatomy and/or a bodily/psychological process of the patient, wherein the virtual model uses input data, providing characteristics of the patient, to generate output data.

The method 300 comprises a step 302 of obtaining treatment information 360 for each of a plurality of different possible treatment strategies.

The virtual model and/or the treatment information may be obtained from a database, data storage arrangement, remote server or memory 355, 365 that stores the virtual model and/or treatment information. In some examples, the treatment information is generated by the virtual model itself. In other examples, the treatment information is selected by a user (e.g. via a user interface).

The method 300 further comprises a step 303 of, for each treatment strategy, determining the effect of that treatment strategy on a virtual model. This step may be performed in an analogous manner to any previously described method (e.g. with reference to previously described step 103).

The method 300 further comprises a step 304 of generating effect information 370 for each treatment strategy. This may be performed in an analogous manner to previously described methods (e.g. with particular reference to previously described step 104).

Steps 303 and 304 may be conceptually combined into a single step of processing, for each of the plurality of possible treatment strategies, the corresponding treatment information and the virtual model to generate, for each of the plurality of possible treatment strategies, effect information that indicates the predicted effect of the treatment strategy on the virtual model of the patient.

The method further comprises an optional step 305 of generating comparative information for comparing the treatment strategies based on the effect information generated in step 304.

Step 305 may comprise, for example, ranking each treatment strategy based on the effect information generated in step 304. When scoring and ranking the treatment candidates, it would be preferred that the patient's health is considered as a main factor. As previously explained, the ongoing ability to monitor the patient's health may rely upon the suitability of the virtual model, which is therefore an important consideration in ranking a treatment candidate with respect to their health. In other words, the candidates are ranked from the most suitable to the least suitable for the patient. Step 305 may comprise performing any other comparative analysis process on the treatment strategies based on the effect information of each treatment strategy.

For example, step 305 may comprise clustering the treatment strategies based on the effect information of each strategy, to cluster those having similar effects together. As another example, step 305 may comprise comparing treatment strategies based on their effect information to determine pros and cons of each treatment strategy based on the effect information.

Other methods of comparing treatment strategies using effect information, to thereby generate comparative information, will be apparent to the skilled person.

The method may further comprise a step 306 of displaying, e.g. at a user interface, the comparative information.

For example, where the comparative information comprises a rank of each treatment strategy, this step may comprise displaying a rank of each treatment strategy or otherwise indicating how each treatment strategy is ranked with respect to the other treatment strategies.

Where the comparative information comprises clustering information of the treatment strategies (e.g. generated by a clustering process) the display may indicate to which cluster a particular treatment strategy belongs or pictorially display the clusters of treatment strategies.

Step 306 may be performed using any suitable user interface, such as a (touch-sensitive) screen or display, e.g. a two-dimensional LCD or LED screen.

These embodiments provide a more useful tool for a clinician in making a clinical decision on which treatment strategy is most suitable for the long-term health of the patient.

The skilled person would appreciate that whilst proposed embodiments discuss only a single virtual model, any described method can be adapted for a plurality of virtual models (e.g. by determining the effect on each of a plurality of virtual models). Thus, effect information may be generated for each of a plurality of virtual models for the patient. This may be beneficial so that a clinician can identify the effect of a particular treatment option on different virtual models of the patient (e.g. even if the treatment is attempting to solve a problem unrelated to a particular virtual model).

In some such embodiments, the “plurality of virtual models” may comprise virtual models associated with known problem areas of the patient (e.g. areas currently being monitored). Thus, a step of obtaining a plurality of virtual models may comprise obtaining patient data, determining one or more problem areas of the subject based on the patient data (e.g. areas in which the patient is experiencing symptoms or areas for which the patient is receiving treatment) and obtaining virtual models associated with the one or more problem areas of the subject (e.g. from a database or the like).

Virtual models that may be employed in the present invention are well known in the art. In some examples, a virtual model may be implemented using a machine-learning algorithm and/or one or more biophysical models (e.g. mathematical models) that receive input data (e.g. patient parameters and/or characteristics) to generate output data (e.g. predicted patient characteristics, diagnoses, treatment options and so on).

A machine-learning algorithm is any self-training algorithm that processes input data in order to produce or predict output data. Here, the machine-learning algorithm is employed to simulate or model a bodily or psychological process of a patient and/or part of the anatomy of the patient.

Suitable machine-learning algorithms for being employed in the present invention will be apparent to the skilled person. Examples of suitable machine-learning algorithms include decision tree algorithms and artificial neural networks. Other machine-learning algorithms such as logistic regression, support vector machines or Naïve Bayesian model are suitable alternatives.

The structure of an artificial neural network (or, simply, neural network) is inspired by the human brain. Neural networks are comprised of layers, each layer comprising a plurality of neurons. Each neuron comprises a mathematical operation. In particular, each neuron may comprise a different weighted combination of a single type of transformation (e.g. the same type of transformation, sigmoid etc. but with different weightings). In the process of processing input data, the mathematical operation of each neuron is performed on the input data to produce a numerical output, and the outputs of each layer in the neural network are fed into the next layer sequentially. The final layer provides the output.

Methods of training a machine-learning algorithm are well known. Typically, such methods comprise obtaining a training dataset, comprising training input data entries and corresponding training output data entries. An initialized machine-learning algorithm is applied to each input data entry to generate predicted output data entries. An error between the predicted output data entries and corresponding training output data entries is used to modify the machine-learning algorithm. This process can repeated until the error converges, and the predicted output data entries are sufficiently similar (e.g. ±1%) to the training output data entries. This is commonly known as a supervised learning technique.

For example, where the machine-learning algorithm is formed from a neural network, (weightings of) the mathematical operation of each neuron may be modified until the error converges. Known methods of modifying a neural network include gradient descent, backpropagation algorithms and so on.

The training input data entries correspond to example entries of input data. The training output data entries correspond to corresponding example entries of output data.

A biophysical model may be a mathematical model derived from patient information that can model at least part of the anatomy and/or a bodily/psychological process of the patient.

Baillargeon, B., Rebelo, N., Fox, D. D., Taylor, R. L., & Kuhl, E. (2014). The Living Heart Project: A robust and integrative simulator for human heart function. European journal of mechanics. A, Solids, 48, 38-47. doi:10.1016/j.euromechso1.2014.04.001 provides one example of developing a biophysical model.

In this example, computer tomography and magnetic resonance images can be processed to create an anatomic model of the human heart. Similarly, computer tomography and magnetic resonance images can be processed to derive a circulatory model of the human heart.

Electro-mechanical coupling of heart parts and their functions can be modelled mathematically based on kinematic equations, the balance equations, the constitutive equations of excitation-contraction coupling. Subsequently, a finite element computation model is generated by combining the models of the electro-mechanical coupling of heart parts and their function.

Finally, combing all of above elements, a human heart model can be created, which includes a solid model, a finite element model, a muscle fiber model, fluid model and so on. Using these models, it is possible to simulate and/or analyze the spatio-temporal evolution of electrical potential, mechanical deformation, muscle fiber strain and so on.

A similar approach may be adopted for generating a biophysical model for any other aspect of the patient.

A combination of machine-learning algorithms and/or biophysical models may be used to construct the virtual model. In some examples, a biophysical model is at least partially generated using one or more machine-learning algorithm (e.g. to perform the creation of the anatomic model of the heart based on images).

Some embodiments may comprise retraining or further training the virtual model. In embodiments in which the virtual model comprises a machine-learning algorithm, this may be performed by obtaining additional training information (e.g. from a database) and further training the virtual model. In embodiments in which the virtual model comprise a biophysical model, it is possible to modify parameters of the biophysical model using a machine-learning training approach.

The skilled person would be readily capable of developing a processing system for carrying out any herein described method. Thus, each step of the flow chart may represent a different action performed by a processing system, and may be performed by a respective module of the processing system.

Embodiments may therefore make use of a processing system. The processing system can be implemented in numerous ways, with software and/or hardware, to perform the various functions required. A processor is one example of a processing system that employs one or more microprocessors that may be programmed using software (e.g., microcode) to perform the required functions. A processing system may however be implemented with or without employing a processor, and may be implemented as a combination of dedicated hardware to perform some functions and a processor (e.g., one or more programmed microprocessors and associated circuitry) to perform other functions.

Examples of processing system components that may be employed in various embodiments of the present disclosure include, but are not limited to, conventional microprocessors, application specific integrated circuits (ASICs), and field-programmable gate arrays (FPGAs).

In various implementations, a processor or processing system may be associated with one or more storage media such as volatile and non-volatile computer memory such as RAM, PROM, EPROM, and EEPROM. The storage media may be encoded with one or more programs that, when executed on one or more processors and/or processing systems, perform the required functions. Various storage media may be fixed within a processor or processing system or may be transportable, such that the one or more programs stored thereon can be loaded into a processor or processing system.

It will be understood that disclosed methods are preferably computer-implemented methods. As such, there is also proposed the concept of computer program comprising code means for implementing any described method when said program is run on a processing system, such as a computer. Thus, different portions, lines or blocks of code of a computer program according to an embodiment may be executed by a processing system or computer to perform any herein described method. In some alternative implementations, the functions noted in the block diagram(s) or flow chart(s) may occur out of the order noted in the figures. For example, two blocks shown in succession may be executed substantially concurrently, or the blocks may sometimes be executed in the reverse order, depending upon the functionality involved.

Variations to the disclosed embodiments can be understood and effected by those skilled in the art in practicing the claimed invention, from a study of the drawings, the disclosure and the appended claims. In the claims, the word “comprising” does not exclude other elements or steps, and the indefinite article “a” or “an” does not exclude a plurality. A single processor or other unit may fulfill the functions of several items recited in the claims. The mere fact that certain measures are recited in mutually different dependent claims does not indicate that a combination of these measures cannot be used to advantage. If a computer program is discussed above, it may be stored/distributed on a suitable medium, such as an optical storage medium or a solid-state medium supplied together with or as part of other hardware, but may also be distributed in other forms, such as via the Internet or other wired or wireless telecommunication systems. If the term “adapted to” is used in the claims or description, the term “adapted to” is intended to be equivalent to the term “configured to”. Any reference signs in the claims should not be construed as limiting the scope.

1. A computer-based method of predicting the effect of a potential treatment strategy on a virtual model of a patient, the computer-based method comprising:
obtaining a virtual model of a patient, the virtual model comprising a digital representation of at least part of the anatomy and/or a bodily/psychological process of the patient, wherein the virtual model uses input data, providing information on characteristics of the patient, to generate output data;
receiving treatment information, the treatment information indicating a potential treatment strategy for the patient;
processing the treatment information and the virtual model to predict an effect of the potential treatment strategy on the virtual model of the patient; and
generating, based on the processing, effect information indicates the predicted effect of the potential treatment strategy on the virtual model of the patient.

2. The computer-based method of claim 1, wherein the step of receiving treatment information comprises using the virtual model to generate the treatment information.

3. The computer-based method of any of claim 2, wherein the step of processing the treatment information and the virtual model comprises predicting the effect of the treatment information on the input data used by the virtual model, to thereby predict the effect of the treatment information on the virtual model.

4. The computer-based method of claim 3, wherein the step of predicting the effect of the treatment information on the input data comprises predicting:
the effect of the potential treatment strategy on the availability of at least some of the input data;
the effect of the potential treatment strategy on noise or error-rate in at least some of the input data; and/or
whether the potential treatment strategy will introduce new features to the input data.

5. The computer-based method of claim 1, wherein the step of processing the treatment information and the virtual model comprises:
using the virtual model to generate first output data based on the input data;
modifying the input data based on the treatment information;
using the virtual model to generate second output data based on the modified input data; and
comparing the first output data and the second output data to predict the effect of the treatment information on the virtual model.

6. The computer-based method of claim 5, wherein:
the step of comparing the first output data to the second output data comprises predicting an accuracy of the virtual model, after the treatment strategy is performed on the patient, using the first output data and the second output data; and
the step of generating effect information comprises outputting the determined accuracy as the effect information.

7. The computer-based method of any of claim 5, wherein the first output data comprises a first recommended treatment strategy and the second output data comprises a second recommended treatment strategy.

8. The computer-based method of any of claim 1, wherein the step of processing the treatment information and the virtual model comprises predicting the effect of the treatment information on the step of generating output data performed by the virtual model, to thereby predict the effect of the treatment information on the virtual model.

9. The computer-based method of claim 8, wherein the step of processing the treatment information and the virtual model comprises predicting the effect of the treatment information on the output data generated by the virtual model, to thereby predict the effect of the treatment information on the virtual model.

10. The computer-based method of claim 9, wherein the step of processing the treatment information and the virtual model comprises:
predicting, based on the treatment information, a physiological effect of the potential treatment strategy on one or more physiological parameters of the patient or on the monitoring of one or more physiological parameters; and
determining, based on the predicted physiological effect and the virtual model, the effect of the potential treatment strategy on the virtual model of the patient.

11. The computer-based method of claim 10, wherein the step of processing the treatment information and the virtual model comprises:
predicting, based on the treatment information, a behavioral and/or psychological effect of the potential treatment strategy on the patient and/or caregivers; and
determining, based on the predicted behavioral and/or psychological effect and the virtual model, the effect of the potential treatment strategy on the virtual model of the patient.

12. The computer-based method of claim 11, further comprising a step of receiving patient data, the patient data providing information on the patient, and wherein the step of processing the treatment information and the virtual model comprises processing the treatment information, the patient data and the virtual model to predict a combined effect of the treatment information and the patient data on the virtual model of the patient.

13. A computer-based method of generating comparative information for comparing treatment strategies for a patient, the computer-based method comprising:
obtaining a virtual model of a patient, the virtual model comprising a digital representation of at least part of the anatomy and/or a bodily/psychological process of the patient, wherein the virtual model uses input data, providing information on characteristics of the patient, to generate output data;
obtaining treatment information for each of a plurality of different possible treatment strategies;
processing, for each of the plurality of possible treatment strategies, the corresponding treatment information and the virtual model to generate, for each of the plurality of possible treatment strategies, effect information that indicates the predicted effect of the treatment strategy on the virtual model of the patient; and
generating comparative information based on the effect information generated by processing each treatment strategy.

14. The computer-based method of claim 13, wherein the step of generating comparative information comprises ranking the treatment strategies based on the effect information generated by processing each treatment strategy.

15. The computer-based method of claim 13, wherein the step of processing comprises, for each of the plurality of possible treatment strategies:
processing the corresponding treatment information and the virtual model to predict an effect of the treatment information on the virtual model of the patient; and
generating, based on the processing, effect information that indicates the predicted effect of the treatment information on the virtual model of the patient.

16. The computer-based method of claim 15, wherein the step of processing the treatment information and the virtual model comprises predicting the effect of the treatment information on the input data used by the virtual model, to thereby predict the effect of the treatment information on the virtual model.

17. The computer-based method of claim 16, wherein the step of predicting the effect of the treatment information on the input data comprises predicting:
the effect of the potential treatment strategy on the availability of at least some of the input data;
the effect of the potential treatment strategy on noise or error-rate in at least some of the input data; and
whether the potential treatment strategy will introduce new features to the input data.

18. The computer-based method of claim 15, wherein the step of processing the treatment information and the virtual model comprises:
using the virtual model to generate first output data based on the input data;
modifying the input data based on the treatment information;
using the virtual model to generate second output data based on the modified input data; and
comparing the first output data and the second output data to predict the effect of the treatment information on the input data.

19. The computer-based method of claim 18, wherein:
the step of comparing the first output data to the second output data comprises predicting an accuracy of the virtual model, after the treatment strategy is performed on the patient, using the first output data and the second output data; and
the step of generating effect information comprises outputting the determined accuracy as the effect information.

20. The computer-based method of claim 19, wherein the first output data comprises a first recommended treatment strategy and the second output data comprises a second recommended treatment strategy.

21. The computer-based method of claim 20, wherein the step of processing the treatment information and the virtual model comprises predicting the effect of the treatment information on the step of generating output data performed by the virtual model, to thereby predict the effect of the treatment information on the virtual model.

22. The computer-based method of claim 21, wherein the step of processing the treatment information and the virtual model comprises predicting the effect of the treatment information on the output data generated by the virtual model, to thereby predict the effect of the treatment information on the virtual model.

23. The computer-based method of claim 22, further comprising a step of receiving patient data, the patient data providing information on the patient, and wherein the step of processing the treatment information and the virtual model comprises processing the treatment information, the patient data and the virtual model to predict a combined effect of the treatment information and the patient data on the virtual model of the patient.

24. A computer program product comprising computer program code that, when executed on a computing device having at least one processing system, causes the at least one processing system to perform all of the steps of the method according to claim 1.

25. A processing system adapted to:
obtain a virtual model of a patient, the virtual model comprising a digital representation of at least part of the anatomy and/or a bodily/psychological process of the patient, wherein the virtual model uses input data, providing information on characteristics of the patient, to generate output data;
obtain treatment information for each of a plurality of different possible treatment strategies;
process, for each of the plurality of possible treatment strategies, the corresponding treatment information and the virtual model to generate, for each of the plurality of possible treatment strategies, effect information that indicates the predicted effect of the treatment strategy on the virtual model of the patient; and
generate comparative information based on the effect information generated by the step of evaluating each treatment strategy.
